(19) United States
(12) Patent Application Publication
(10) Pub. No.: US 2026/0256990 A1
(43) Pub. Date: Sep. 3, 2026

(54) INJECTABLE HYDROGEL MATERIAL FORMED IN-SITU AND USE THEREOF IN THE PREPARATION OF TISSUE REPAIR FORMULATIONS

(71) Applicant: SHENZHEN ZHIHUI MEDICAL TECHNOLOGY CO., LTD, Guangdong (CN)

(72) Inventors: Yulong SUN, Guangdong (CN); Bing LIU, Guangdong (CN); Wenhua LIANG, Guangdong (CN)

(73) Assignee: SHENZHEN ZHIHUI MEDICAL TECHNOLOGY CO., LTD, Guangdong (CN)

(21) Appl. No.: 19/653,007

(22) Filed: Apr. 20, 2026

Related U.S. Application Data
(63) Continuation of application No. PCT/CN2024/098740, filed on Jun. 12, 2024.

(30) Foreign Application Priority Data
Oct. 27, 2023 (CN) 202311425112.7

(51) Int. Cl. A61L 27/52; A61L 27/44

(52) U.S. Cl. CPC A61L 27/52; A61L 27/44; A61L 2400/06; A61L 2430/30; A61L 2430/40

(57) ABSTRACT

An injectable hydrogel material formed in-situ of the present invention includes two fluid components: component A and component B, wherein component A is an activated linker solution, and component B is a collagen and/or gelatin fluid. The hydrogel material of the present invention is prepared in use. First, a cross-linking activator is used to activate the linker to give component A. During this process, a portion of the cross-linking activator is consumed, thus reducing the amount of cross-linking activator in contact with tissues. Subsequently, component A and component B are mixed, and the macromolecules in the components undergo chemical cross-linking to form a gel. A neutralizing agent is introduced into component B to consume the cross-linking activator in the material during the chemical cross-linking of the macromolecules and a period thereafter.

### CROSS-REFERENCE TO RELATED APPLICATION

This application is a continuation of PCT application No. PCT/CN2024/098740, filed on Jun. 12, 2024, which claims the priority benefit of China application no. 202311425112.7, filed on Oct. 27, 2023. The entirety of each of the above mentioned patent applications is hereby incorporated by reference herein and made a part of this specification.

### TECHNICAL FIELD

The present invention relates to the technical field of medical biomaterials, and more specifically, to an injectable hydrogel material formed in-situ and use thereof in the preparation of tissue repair formulations.

### RELATED ART

In daily life, human tissues suffer various injuries, resulting in tissue disruption or defects. Injured tissues can be repaired and restored through regeneration and reconstruction. The process of tissue repair includes the collection of cells from the damaged tissue and other parts of the body to the injured site to undergo division, proliferation, and extracellular matrix reconstruction. The living environment is very important for cells to carry out the above-mentioned tasks, and any factors, which affect the cell's living environment, could affect tissue healing. For a long time, people have been exploring various therapeutic methods, biomaterial products, and drugs to shorten tissue healing time, especially for the repair of chronic and difficult-to-heal tissue injuries.

Collagen mainly exists in mammalian tissues, such as skin, tendons, cartilage, and bone, with abundant content, accounting for about 20%-30% of the total protein in the human body or other animals. As a major component of the mammalian extracellular matrix, collagen participates in the formation of the three-dimensional spatial network around cells, playing an important role in maintaining the structural integrity of the extracellular matrix and the biological functions of cells. Collagen and its partially hydrolyzed product, gelatin, possess the arginine-glycine-aspartic acid (RGD) sequence, and exhibit excellent biological activities in promoting cell adhesion, differentiation, and growth. They possess a number of advantages, such as excellent biocompatibility, weak antigenicity, degradability, and safety, and are frequently used as raw materials to prepare biomedical products for guiding tissue regeneration and promoting wound healing. Medical products developed using collagen or gelatin as key materials include double-layer artificial dermal repair materials, medical collagen repair membranes, surgical biological patches, artificial nerve conduits, and cartilage scaffolds. The materials composed sole collagen or gelatin have rapid biodegradation rate and low mechanical strength, which make the materials less effective to promote the regeneration of injured tissues. Furthermore, the gels composed of gelatin have a relatively low melting point and cannot coagulate at body temperature. Therefore, the medical products, in which collagen or gelatin serves as a key component, the collagen and gelatin generally need to be crosslinked through physical, chemical, or enzymatic methods to form a stable three-dimensional network stable structure with a certain mechanical strength. Although the crosslinking with genipin or transglutaminase has good biocompatibility, the crosslinking time is long, and the mechanical strength of cross-linked products are weak. The use of formaldehyde, glutaraldehyde, carbodiimide, etc., which have poor biocompatibility, as crosslinking agents or crosslinking activators, results in a short crosslinking time and good mechanical strength of the cross-linked products. However, the residual cross-linking agents/cross-linking activators in the materials can be irritating or toxic to cells and tissues, which can have adverse effects on tissue repair. Therefore, during product preparation, residual crosslinking agents/crosslinking activators are usually removed from the material by the methods, such as washing or dialysis, after crosslinking is completed.

With the development of technology, minimally invasive surgery has been widely used in clinical practice. Solid medical material products, such as sponges, films and molded gels, are suitable for the exposed and easily manipulated wounds, but are difficult to be applied in minimally invasive and endoscopic surgeries. Therefore, the novel medical material products need to be developed. Under physiologically mild conditions, the novel medical material products or raw materials are necessary to be delivered to the injury sites through injection or spraying, rapidly formed, have a certain mechanical strength, and remain in the body for a certain period of time.

Many medical products, in which collagen or gelatin is the key component, are prepared in vitro using physical, enzymatic, and/or chemical crosslinking methods. These traditional preparation methods are not suitable for in vivo use. Although some chemical crosslinking methods can be used under physiological conditions, the rapid crosslinking of collagen or gelatin using traditional methods requires the high concentration of crosslinking agents/crosslinking activators. Residual crosslinking agents/crosslinking activators could damage cells, irritate tissues, and limit tissue repair. The methods, such as washing or dialysis, to remove residual crosslinking agents/crosslinking activators from in vivo materials are time-consuming, laborious, ineffective, and difficult to be applied in minimally invasive surgery.

Disclosed in the prior art are a hyaluronic acid-gelatin-acrylamide dual-network hydrogel and a preparation method therefor, specifically disclosing the following: (1) Preparation of modified hyaluronic acid: a. Prepare a hyaluronic acid aqueous solution with a mass fraction of 1%-4%, the pH of the hyaluronic acid aqueous solution to 4-6 with hydrochloric acid, then add 1-(3-dimethylaminopropyl)-3-ethylcarbodiimide and N-hydroxysuccinimide. The mixture was stirred at room temperature for 15-25 min, then cysteine ethyl ester hydrochloride was added. The reaction was carried on for 4-8 hours to obtain a solution. The molar ratio of cysteine ethyl ester hydrochloride to N-hydroxysuccinimide to 1-(3-dimethylaminopropyl)-3-ethylcarbodiimide to hyaluronic acid was 1-2:0.5-1:0.25-1:1; b. Add methacrylic anhydride to the solution obtained in Step a. The pH of solution was adjusted to 7.0-9.0 with a NaOH solution. The reaction was performed at 2° C.-6° C. for 20-48 hours. The reactant was dialysed for 5-7 days, then lyophilized to obtain a solid of modified hyaluronic acid. The molar ratio of the methacrylic anhydride to the hyaluronic acid in Step a is 1-10:1; (2) Preparation of a first network hydrogel: the modified hyaluronic acid obtained in step (1) was dissolved in an aqueous solution, then mixed with a gelatin aqueous solution and a photoinitiator. The mixture was stirred at room temperature in the dark for 20-30 min, and irradiated with ultraviolet light for 20-40 min to form the first network hydrogel. The photoinitiator was either 2-hydroxy-4′-(2-hydroxyethoxy)-2-methylphenylacetone or α-ketoglutaric acid. The concentration of the modified hyaluronic acid in aqueous solution was 1-4 g/100 mL; the concentration of gelatin was 2-10 g/100 mL; the volume ratio of the modified hyaluronic acid solution to the gelatin solution was 4:1-16; the mass ratio of the photoinitiator to the modified hyaluronic acid was 40:1-6. The above preparation steps of the hyaluronic acid-gelatin-acrylamide dual-network hydrogel material are complex. The process of dialysis was required 5-7 days, which resulted in a long overall reaction time. Furthermore, the reaction procedure cannot be performed in situ, which obviously cannot meet the above application requirements for in vivo tissue repair and reconstruction.

### SUMMARY OF INVENTION

An objective of the present invention is to overcome the defects and shortcomings of high residual amount of crosslinking agents/crosslinking activators, complex and time-consuming elimination methods, and the products' inability to coagulate in situ in the existing medical biomaterials for tissue repair. The present invention provides an injectable hydrogel material formed in-situ, containing an activated linker solution (component A), and a fluid containing collagen/gelatin (component B). Before the material contacts the tissue, the activated linker has consumed a portion of the crosslinking activator, and has reduced the amount of crosslinking activator in contact with the tissue. Furthermore, during and after the mixing of the component A and the component B, the neutralizing agent introduced in the component B can consume the crosslinking activator at the same time of chemical cross-linking the component macromolecules and within a subsequent period of time, further reducing the residual amount of the crosslinking activator in the material mixture in contact with the tissue, and improving the biocompatibility of the material. The procedure of material preparation is simple, the gelation time is short, and the hydrogel material is capable of injection and coagulates in situ.

Another objective of the present invention is to provide the use of an injectable hydrogel material formed in-situ for the preparation of tissue repair formulations.

Yet another objective of the present invention is to provide a tissue repair formulation.

The above objectives of the present invention are implemented through the following technical solutions:

An injectable hydrogel material formed in-situ, including two fluid components: component A and component B, wherein the component A is an activated linker solution with a pH value of 4-8, and the component B is a collagen and/or gelatin fluid with a pH value of 4-9, and
the component A and the component B exist independently before use, and during use, the component A and the component B are mixed and chemically cross-linked to form a gel material.

In the present invention, the component A is preferably an activated linker. Before it is extruded and mixed with the component B to form a gel material in contact with the tissue, a portion of the crosslinking activator is consumed, thereby reducing the amount of the crosslinking activator in the material in contact with the tissue.

In the injectable hydrogel material formed in-situ of the present invention, the pH values of the component A and the component B affect the efficiency of chemical coupling after final mixing, which concerns injection and coagulation of the hydrogel material.

After the crosslinking of the raw macromolecule materials in the component A and the component B is completed, there may still be the crosslinking activators in the material. Neutralizing agents is used to continually remove the crosslinking activators, especially those without carboxyl groups.

It Needs to be Noted that:

The present invention introduces a linker with good biocompatibility to improve the crosslinking among macromolecules in the material. Even if a low concentration of crosslinking activator is used, the gel material exhibits good stability and strength.

The main components in the injectable hydrogel material formed in-situ of the present invention are collagen and/or gelatin. Collagen is derived from mammals, fish, or genetic engineering. The mammals are preferably cattle and pigs. The production of genetically engineered collagen involves recombinant collagen genes expressed in bacteria, yeast, cells, insects, or transgenic crops. The collagen includes one or more types, such as type I, type II, and type III, and is preferably a telopeptide-depleted collagen.

Gelatin is a partially hydrolyzed product of collagen. It can be of animal or genetic engineering origin. Animal gelatin is extracted by acid treatment (type A gelatin) or alkali treatment (type B gelatin). The gel strength of gelatin is in the range of 100-400 Bloom, and gelatin with a gel strength not less than 200 Bloom is preferred.

The chemical crosslinking of the injectable hydrogel material formed in-situ of the present invention is prepared during use; the component A and the component B are uniformly mixed, and the volume ratio of the component A to the component B is 10:1 to 1:10, preferably 1:1. The start time for mixing components A and B is at least 5 min from the mixing time of the linker and crosslinking activator, preferably 10-100 min.

Most of the existing biomaterial products have been prefabricated before use, and are used in a form of solid or gel. The molded biomaterials are difficult to implant into the body through minimally invasive and interventional methods, and are difficult to conveniently place in the injured or diseased sites. The injectable hydrogel material formed in-situ of the present invention is not prefabricated. The raw materials are first mixed and prepared into two fluids: component A and component B. Then, the prepared component A and component B are injected into the tissue damage site. During injection and/or within a short time after reaching the tissue damage site, they are mixed uniformly and quickly coagulate. Therefore, the injectable hydrogel material of the present invention can achieve in-situ forming and is suitable not only for the exposed and easily manipulated wounds, but also for the minimally invasive and interventional surgeries.

When the injectable hydrogel material formed in-situ of the present invention reaches the damage site, it is not fully cross-linked and remains in a flowing state. Therefore, it can fit with the various wounds, including uneven wounds and cavity wounds.

Furthermore, the molecular framework of the injectable hydrogel material formed in-situ in the present invention is composed of collagen/gelatin and a linker. The collagen is a right-handed superhelix formed by the intercrossing of three left-handed helical polypeptide chain molecules along a common axis. Gelatin is a product of partial hydrolysis/unwinding of collagen. Each polypeptide molecule of collagen contains multiple amino and carboxyl groups. The linker is a biomacromolecule with multiple carboxyl groups. The crosslinking activator carbodiimide can promote the chemical reaction between carboxyl group and primary amino group to form amide bond. Therefore, the molecular network of the invented gel material includes self-crosslinking (intramolecular and intermolecular) of collagen/gelatin and crosslinking of collagen/gelatin with the linker. Usually, collagen/gelatin-based biomaterials are prepared by using a one-step synthesis method, which the crosslinking agent/crosslinking activator is directly mixed with collagen/gelatin and other raw material solutions. The gel material of the present invention adopts a two-step synthesis method, which the linker is first activated by reacting with the crosslinking activator (component A), and then component A is mixed with collagen/gelatin in component B containing a neutralizing agent. The neutralizing agent can reduce the amount of the crosslinking activator in the material. Compared with the one-step synthesis method, the two-step synthesis method used in the present invention can more effectively activate the linker, reduce the self-crosslinking of collagen/gelatin, increase the crosslinking between the linker and collagen/gelatin, and form a molecular network structure with a mode of crosslinking different from that of the one-step synthesis method.

The gel of the present invention not only can bind to tissues through hydrogen bonds and ionic bonds, but also can form covalent bond with amino groups on the surface of tissue through the activated carboxyl groups in collagen/gelatin and the linker, thereby enhancing the binding strength between the gel and tissue.

In a specific embodiment, the linker in the component A of the present invention includes any one or mixture of hyaluronic acid, alginate, sodium carboxymethyl cellulose, sodium carboxymethyl starch, and derivatives thereof, and the linker has a mass content of 0.01%-5% in the injectable hydrogel material.

The crosslinking activator of linker in the component A includes 1-ethyl-3-(3-dimethylaminopropyl) carbodiimide (EDC) or a hydrochloride salt thereof, and the crosslinking activator has a mass content of 0.1%-3% in the injectable hydrogel material.

The injectable hydrogel material formed in-situ of the present invention uses collagen and/or gelatin, which can guide tissue regeneration and promote wound healing, as the main raw materials. The use of EDC or a hydrochloride salt thereof, which has relatively low toxicity/irritation, as a crosslinking agent to reduce the irritation and toxicity that tissues may exposed to from the aspect of raw materials.

The linker is a macromolecule that connects the peptide chains of collagen/gelatin in the gel and plays a role to enhance the stability of the gel material. It is a biomacromolecule containing multiple carboxyl groups, including hyaluronic acid, alginate, sodium carboxymethyl cellulose, sodium carboxymethyl starch, and derivatives thereof.

The crosslinking activators are molecules that promote the chemical crosslinking of collagen/gelatin and the linkers, including water-soluble carbodiimide, such as 1-ethyl-3-(3-dimethylaminopropyl) carbodiimide (EDC) or hydrochloride salt thereof, which reacts with carboxyl group to form an O-acylurea active intermediate. The intermediate then reacts with a primary amino group to form an amide bond.

In specific embodiments, the crosslinking activator of the present invention, 1-ethyl-3-(3-dimethylaminopropyl) carbodiimide hydrochloride, can be used alone or in combination with other crosslinking enhancers, such as N-hydroxysuccinimide (NHS) and/or N-hydroxythiosuccinimide (Sulfo-NHS), to improve the efficiency of amide bond formation and the crosslinking efficiency of collagen/collagen peptide chains and the linker, and to reduce the concentration of the crosslinking activator used.

In the specific embodiment, the content of 1-ethyl-3-(3-dimethylaminopropyl) carbodiimide hydrochloride in the gel material is 0.1%-3%, and the molar amount of NHS or Sulfo-NHS is 0-2 times that of carbodiimide.

In the specific embodiment, the component A of the present invention is preferably prepared according to the following method:

A linker and a crosslinking activator are dissolved in a carboxyl-free solvent, which the pH value has been adjusted between 4 and 8. The component A is obtained with the activation reaction time for ≥5 min.

The mass concentration of the linker in the component A is 0.01%-10%.

The linker solution can be prepared in advance or prepared extemporaneously before use.

The above-mentioned linker and crosslinking activator can be dissolved in the following methods:

The crosslinking activator is directly dissolved in the linker solution,

Alternatively, the crosslinking activator is first dissolved in a carboxyl-free solvent, which can be the same as or different from the solvent used to dissolve the linker, and then mixed with the linker solution.

The activation time is preferably 10-120 min.

The weight ratio of the crosslinking activator, 1-ethyl-3-(3-dimethylaminopropyl) carbodiimide hydrochloride, to the linker is 10:1 to 1:10, preferably 5:1 to 1:5.

The carboxyl-free solvents of the present invention may include any one of the following solvents:

distilled water, physiological saline, dilute hydrochloric acid solution, phosphate buffer solution (PBS), and 2-morpholinoethanesulfonic acid (MES) buffer.

The pH of PBS is 5.8-8.0, and the pH of the MES buffer is 5.4-6.8.

Collagen and/or gelatin in the component B of the present invention are dissolved or suspended in an aqueous solution to form an injectable fluid. Collagen has a mass content of 0.1%-15% in the injectable hydrogel material, and gelatin has a mass content of 1%-30% in the injectable hydrogel material.

In specific embodiments, the aqueous solution is preferably an aqueous solution containing the component with carboxyl group.

In the technical solution of the present invention, the component B also includes a neutralizing agent for reducing the amount of unreacted carbodiimide in the gel material, thereby reducing the amount of carbodiimide in contact with the tissue.

The neutralizing agent is an organic molecule containing carboxyl group or an organic molecule capable of generating carboxyl group. In embodiments of the present invention, the neutralizing agent may be any one or combination of formic acid, acetic acid, propionic acid, oxalic acid, malonic acid, ethylenediaminetetraacetic acid, ascorbic acid, citric acid, lipoic acid, glutamic acid, aspartic acid, glutathione, polyglutamic acid, polyaspartic acid, and derivatives thereof.

It Needs to be Noted that:

The organic molecules that can generate carboxyl group in the present invention refer to molecules that do not contain carboxyl group themselves, but generate the product with carboxyl group after reactions. The reactions could be hydrolysis, oxidation, enzyme reaction, etc.

### The Neutralizing Agent can be Introduced by the Following Two Methods:

Method 1: The molecules are used to regulate the pH of the aqueous solution for dissolving or suspending collagen/gelatin in the present invention, for example, any one of acetic acid solution, acetic acid-sodium acetate buffer, citric acid-sodium citrate buffer, and citric acid-disodium hydrogen phosphate buffer.

The pH of the acetic acid-sodium acetate buffer is 3.6-5.8, the pH of the citric acid-sodium citrate buffer is 3.0-6.6, and the pH of the citric acid-disodium hydrogen phosphate buffer is 2.2-8.0.
Method 2: organic molecules containing carboxyl group or organic molecules capable of generating carboxyl group, are simply used for reducing the amount of unreacted carbodiimide in the gel material, wherein the linkers for preparing the component A can also be used as the neutralizing agents.

In specific embodiments, preferably, the molar amount of the carboxyl group or carboxyl group generated from the neutralizing agent in the component B is 0.1-10 times, preferably 0.1-5 times, to that of the activator carbodiimide.

In specific embodiments, the carboxyl-containing buffer and the neutralizing agent could be the same substance, and the linker and the neutralizing agent could also be the same substance.

In addition to collagen/gelatin, the carboxyl-containing neutralizing molecules and/or therefrom the generated carboxyl-containing products in the component B consume the crosslinking activator in the material during and after the mixing of the component A and the component B, further reducing the crosslinking activator in the gel material in contact with the tissue.

The collagen/gelatin solution can be prepared in advance or prepared extemporaneously before use.

The specific preparation method can be the conventional preparation method, such as the following:
1) The gelatin solution is prepared by mixing gelatin with a solvent and heated to 40° C.-80° C. After gelatin is dissolved, a neutralizing agent is added and mixed uniformly. If a carboxyl-containing solvent is used, the neutralizing agent may not be added. Additionally, a linker can also be used as a neutralizing agent. If the pH of the solution is below 4, the pH of the solution is adjusted to 4-9 with an alkali, such as a sodium hydroxide solution. The mass concentration of gelatin is 1%-50%, and the molar amount of the carboxyl group in the neutralizing agent or carboxyl group generated from the neutralizing agent is 0-10 times that of carbodiimide.
2) Collagen solution can be added to the above gelatin solution to prepare a collagen/gelatin solution. If the pH of the solution is below 4, the pH of the solution is adjusted to 4-9 with an alkali, such as a sodium hydroxide solution. The concentration of gelatin is 1%-50%, and the concentration of collagen is 0.1%-10%.
3) Collagen powder, preferably telopeptide-depleted fibrillar collagen powder, can be added to the gelatin solution or collagen solution to prepare a turbid solution. The pH value of the solution is adjusted to 4-9, a neutralizing agent is then added, and the mixture is mixed uniformly and used before solidification. The mass concentration of collagen is 1%-15%.

In specific embodiments, further preferably, the mass content of linker is 0.1%-3% in the injectable hydrogel material.

In specific embodiments, the component B of the present invention further contains a chromogenic agent.

The use of chromogenic agent in the gel can conveniently judge whether the gel is accurately placed at the target position and the coating amount of the gel. It includes toluidine blue, methylene blue, aniline blue, celestine blue, anthocyanin, etc. The mass content of the chromogenic agent in the gel is 0-1%.

This invention also specifically protects the use of the injectable hydrogel material formed in-situ in tissue repair.

The injectable hydrogel material formed in-situ of the present invention can be used to prepare medical gel materials with multiple therapeutic purposes, such as guiding tissue regeneration, promoting wound healing, and preventing tissue leakage and adhesion.

The present invention also specifically protects a tissue repair formulation prepared from the injectable hydrogel material formed in-situ, which is prepared by the following method:

The component A is loaded into a syringe 1. The component B is loaded into a syringe 2. The syringe 1 and the syringe 2 are mounted into two inlets of a three-way connector. Two fluids are simultaneously extruded and uniformly mixed through a mixing tube installed at the outlet of the three-way connector, or by stirred with the outlet of the three-way connector to mix two solutions uniformly, and coagulate to form the tissue repair formulation.

The tissue repair formulation of the present invention can be used to promote tissue healing and regeneration, as well as prevent and treat tissue leakage and adhesion.

For example, it can be used as a medical biomaterial for minimally invasive surgery. After it is implanted into the body by injection during preparation, it rapidly coagulates at the target tissue site.

The tissue repair formulation of the present invention can undergo chemical crosslinking under physiological condition, ensure the excellent biocompatibility of the material, avoid the use of traditional time-consuming and laborious washing or dialysis procedures, and reduce the amount of chemical crosslinking activator in the material in contact with the tissue.

Compared with the prior art, the present invention has the following beneficial effects:

The injectable hydrogel material formed in-situ of the present invention is not prefabricated. During injection and/or after reaching the tissue injury site, it is mixed uniformly in a short time and rapidly coagulates. As the mixture reaches the injury site, it is not fully crosslinked and remains in a flowing state. Therefore, the hydrogel material can fit with various wounds, including wounds with uneven surface and cavity wounds. The hydrogel material is not only suitable for exposed and easily manipulated wounds, but also very suitable for minimally invasive and interventional surgeries, with good tissue conformability.

The two-step synthesis method for the injectable hydrogel material formed in-situ of the present invention can improve the activation efficiency of the linker, reduce the self-crosslinking of collagen/gelatin, increase the crosslinking between the linker and collagen/gelatin, and set up a molecular network structure with a crosslinking mode different from the one-step preparation method. Not only can the material bind to tissues through hydrogen bonds and ionic bonds, but also can form covalent bonds between the activated carboxyl groups in collagen/gelatin/linker and the amino groups on the surface of tissue in contact with the material, so as to enhance the binding strength of the gel and the tissue.

In addition, collagen/gelatin, linker, neutralizing agent, and chromogenic agent, which have good-biocompatibility, are used to prepare the injectable hydrogel material formed in-situ of the present invention. A relatively mild carbodiimide is selected as the crosslinking activator. The linker and the crosslinking enhancer (NHS/Sulfo-NHS) are used in the reaction system to reduce the amount of carbodiimide. Moreover, two-step procedure is adopted for the reaction. Carbodiimide first reacts with the linker to reduce the concentration of carbodiimide in the solution in contact with the tissue. Secondly, the neutralizing agent is included in the component B to reduce the amount of carbodiimide in the material during crosslinking of the raw material macromolecules for a period of time thereafter, further reducing the side effects of the crosslinking activator on cells and tissues and mitigating the irritating effect of carbodiimide on tissues.

The gel material of the present invention takes collagen/gelatin as the main raw materials to bridge tissues separated due to injury, guides cells to repair the injured tissues, thereby promoting tissue healing. Meanwhile, the gel material has the characteristics of good tissue adhesiveness and certain mechanical strength, which can prevent the leakage of tissue contents, including hemostasis. Furthermore, it can isolate the injured tissue from its surrounding tissues, preventing adhesion between the injured tissue and its surrounding tissues.

### BRIEF DESCRIPTION OF DRAWINGS

FIG. 1. Sodium alginate as a linker to improve the stability of a gelatin gel material.

FIG. 2. Cells growing on the surface of a gel material (A: Cultured for 1 day, B: Cultured for 3 days; C: Cultured for 5 days).

FIG. 3. Prevention of tissue adhesion with a gel material (A: Control group, B: Gel group).

FIG. 4. Promotion of tissue healing with a gel material (A1/A2: Abdominal wall muscles without damage; B1/B2: Group without use of material; C1/C2: Group with use of the gel).

### DESCRIPTION OF EMBODIMENTS

The present invention will be further described below in conjunction with specific embodiments, but the embodiments do not limit the present invention in any way. Unless otherwise specified, the raw materials and reagents used in the embodiments of the present invention are conventionally purchased raw materials and reagents.

### Example 1

An injectable hydrogel material formed in-situ, including component A and component B,
wherein the component A and the component B were prepared as follows:

300 mg of sodium alginate was taken and dissolved in 10 ml of a 2-morpholinoethanesulfonic acid (MES) solution (50 mM, pH 6). Then, 200 mg of 1-ethyl-3-(3-dimethylaminopropyl) carbodiimide hydrochloride (EDC) and 200 mg of N-hydroxysuccinimide (NHS) were added. After dissolution, the mixture was allowed to stand for 30 minutes to obtain the component A: an activated sodium alginate solution with a pH value of 4-8;

2 g of gelatin was taken and added to 9 ml of an MES solution (50 mM, pH 6), and the mixture was heated to 60° C. The container was shaken until the gelatin was completely dissolved to prepare the component B: a gelatin solution with a pH value of 4-9.

### Mixing to Form a Gel

The above gelatin solution and activated sodium alginate solution were drawn into two syringes, respectively. The syringes were mounted to a three-way connector with a mixing tube at the outlet. The two syringes were squeezed simultaneously, and the solutions in the two syringes were mixed through the mixing tube at a volume ratio of 1:1. The uniformly mixed solution coagulated to form a gel.

### Example 2

An injectable hydrogel material formed in-situ, including component A and component B,
wherein the component A and the component B were prepared as follows:

200 mg of sodium carboxymethyl cellulose was taken and dissolved in 10 ml of an MES solution (50 mM, pH 6). Then, 200 mg of EDC and 200 mg of NHS were added. After dissolution, the mixture was allowed to stand for 30 minutes to obtain the component A: activated sodium carboxymethyl cellulose with a pH value of 4-8;

2 g of gelatin was taken and added to 9 ml of an MES solution (50 mM, pH 6), and the mixture was heated to 60° C. The container was shaken until the gelatin was completely dissolved. Then, 20 mg of sodium acetate was added. The container was shaken until sodium acetate was completely dissolved, to prepare the component B: a gelatin solution with a pH value of 4-9;

### Mixing to Form a Gel

The above gelatin solution and activated sodium carboxymethyl cellulose solution were drawn into two syringes, respectively. The syringes were mounted to a three-way connector with a mixing tube at the outlet. The two syringes were squeezed simultaneously, and the solutions in the two syringes were mixed through the mixing tube at a volume ratio of 1:1. The uniformly mixed solution coagulated to form a gel.

### Example 3

An injectable hydrogel material formed in-situ, including component A and component B,
wherein the component A and the component B were prepared as follows:

200 mg of sodium hyaluronate was taken and dissolved in 10 ml of an MES solution (50 mM, pH 6). Then, 200 mg of EDC and 200 mg of NHS were added. After dissolution, the mixture was allowed to stand for 30 minutes to obtain the component A: activated sodium hyaluronate with a pH value of 4-8;

2 g of gelatin was taken and added to 9 ml of an acetic acid-sodium acetate buffer (25 mM, pH 5.8), and the mixture was heated to 50° C. for dissolution, to prepare the component B: a gelatin solution with a pH value of 4-9;

### Mixing to Form a Gel

The above gelatin solution and activated sodium hyaluronate solution were drawn into two syringes, respectively. The syringes were mounted to a three-way connector with a mixing tube at the outlet. The two syringes were squeezed simultaneously, and the solutions in the two syringes were mixed through the mixing tube at a volume ratio of 1:1. The uniformly mixed solution coagulated to form a gel.

### Example 4

An injectable hydrogel material formed in-situ, including component A and component B,
wherein the component A and the component B were prepared as follows:

200 mg of sodium alginate was taken and dissolved in 10 ml of physiological saline. Then, 200 mg of EDC and 200 mg of NHS were added. After dissolution, the mixture was allowed to stand for 30 minutes to obtain the component A: an activated sodium alginate solution with a pH value of 4-8;

1 g of gelatin was taken and added to 9.5 ml of physiological saline containing 100 mg of dissolved sodium alginate, and the mixture was heated to 60° C. The container was shaken until the gelatin was completely dissolved. Then, 100 mg of ascorbic acid was added to prepare the component B: a gelatin/sodium alginate solution with a pH value of 4-9;

### Mixing to Form a Gel

The above activated sodium alginate solution and gelatin/sodium alginate solution were drawn into two syringes, respectively. The syringes were mounted to a three-way connector with a mixing tube at the outlet. The two syringes were squeezed simultaneously, and the solutions in the two syringes were mixed through the mixing tube at a volume ratio of 1:1. The uniformly mixed solution coagulated to form a gel.

### Example 5

An injectable hydrogel material formed in-situ, including component A and component B,
wherein the component A and the component B were prepared as follows:

200 mg of sodium hyaluronate was taken and dissolved in 10 ml of PBS (pH 6). Then, 200 mg of EDC and 200 mg of NHS were added. After dissolution, the mixture was allowed to stand for 30 minutes to obtain the component A: an activated sodium hyaluronate solution with a pH value of 4-8;

2 g of gelatin was taken and added to 4 ml of PBS (pH 7.4), and the mixture was heated to 60° C. The container was shaken until the gelatin was completely dissolved, and then 5 ml of telopeptide-depleted bovine collagen acetic acid solution (4 mg/ml) was added. The mixture was mixed uniformly to prepare the component B: a gelatin/collagen solution with a pH value of 4-9;

### Mixing to Form a Gel

The above activated sodium hyaluronate solution and gelatin/collagen solution were drawn into two syringes, respectively. The syringes were mounted to a three-way connector with a mixing tube at the outlet. The two syringes were squeezed simultaneously, and the solutions in the two syringes were mixed through the mixing tube at a volume ratio of 1:1. The uniformly mixed solution coagulated to form a gel.

### Example 6

An injectable hydrogel material formed in-situ, including component A and component B,
wherein the component A and the component B were prepared as follows:

200 mg of sodium alginate was taken and dissolved in 10 ml of an MES solution (50 mM, pH 6). Then, 200 mg of EDC and 200 mg of NHS were added. After dissolution, the mixture was allowed to stand for 30 minutes to obtain the component A: an activated sodium alginate solution with a pH value of 4-8;

1 g of gelatin was taken and added to 9.5 ml of a citric acid-sodium citrate buffer (10 mM, pH 6), and the mixture was heated to 60° C. The container was shaken until the gelatin was completely dissolved, and then 200 mg of telopeptide-depleted bovine fibrillar collagen powder was added. The mixture was mixed uniformly to prepare the component B: a gelatin/collagen suspension with a pH value of 4-9;

### Mixing to Form a Gel

The above activated sodium alginate solution and gelatin/collagen suspension were drawn into two syringes, respectively. The syringes were mounted to a three-way connector with a mixing tube at the outlet. The two syringes were squeezed simultaneously, and the fluids in the two syringes were mixed through the mixing tube at a volume ratio of 1:1. The uniformly mixed fluid coagulated to form a gel.

### Example 7

An injectable hydrogel material formed in-situ, including component A and component B,
wherein the component A and the component B were prepared as follows:

200 mg of sodium carboxymethyl cellulose was taken and dissolved in 10 ml of an MES solution (50 mM, pH 6). Then, 200 mg of EDC and 200 mg of NHS were added. After dissolution, the mixture was allowed to stand for 30 minutes to obtain the component A: an activated sodium carboxymethyl cellulose solution with a pH value of 4-8;

300 mg of telopeptide-depleted bovine fibrillar collagen powder was taken and added to 10 ml of an MES buffer (pH 6) containing 100 mg of dissolved sodium carboxymethyl cellulose (neutralizing agent), then 10 mg of methylene blue was added, and mixed uniformly, to prepare the component B: a collagen/sodium carboxymethyl cellulose suspension with a pH value of 4-9;

### Mixing to Form a Gel

The above collagen/sodium carboxymethyl cellulose suspension and activated sodium carboxymethyl cellulose solution were drawn into two syringes, respectively. The syringes were mounted to a three-way connector with a mixing tube at the outlet. The two syringes were squeezed simultaneously, and the fluids in the two syringes were mixed through the mixing tube at a volume ratio of 1:1. The uniformly mixed fluid coagulated to form a gel.

### Example 8

An injectable hydrogel material formed in-situ, including component A and component B,
wherein the component A and the component B were prepared as follows:

200 mg of sodium hyaluronate was taken and dissolved in 10 ml of an MES solution (50 mM, pH 6). Then, 200 mg of EDC and 200 mg of NHS were added. After dissolution, the mixture was allowed to stand for 30 minutes to obtain the component A: activated sodium hyaluronate with a pH value of 4-8;

300 mg of a fibrillar collagen powder was taken and added to 10 ml of telopeptide-depleted bovine collagen acetic acid solution (4 mg/ml). The mixture was mixed uniformly, and the pH of the solution was adjusted to 6 with a sodium hydroxide solution, to prepare the component B: a collagen suspension with a pH value of 4-9;

### Mixing to Form a Gel

The above collagen suspension and activated sodium hyaluronate solution were drawn into two syringes, respectively. The syringes were mounted to a three-way connector with a mixing tube at the outlet. The two syringes were squeezed simultaneously, and the fluids in the two syringes were mixed through the mixing tube at a volume ratio of 1:1. The uniformly mixed fluid coagulated to form a gel.

### Example 9

This example explored the effect of NHS on EDC to coupling gel raw materials.

In the experiment, 10% (w/w) gelatin solution, 2% sodium alginate solution, 10% EDC solution, and 10% NHS solution were first prepared with use of PBS (pH 6.0); then, the gelatin solution, the sodium alginate solution, and a certain amount of PBS were mixed.

For the formula without NHS, the EDC solution was added, and the final concentrations of gelatin, sodium alginate, and EDC in the mixture were 5%, 0.5%, and 0.3%, respectively.

For the formula with NHS, the EDC solution and the NHS solution were added, and the final concentrations of gelatin, sodium alginate, EDC, and NHS in the mixture were 5%, 0.5%, 0.3%, and 0.3%, respectively.

After the above two mixed solutions were uniformly mixed, the resulting mixed solution was allowed to stand at 37° C. for 2 hours, and the flow state of the mixed solutions was observed. It was found that the mixture without NHS still remained in a flowing state, while the mixture with NHS was in a coagulated state. When the EDC/NHS concentration in the above mixtures decreased to 0.2%, it was also found that the mixture without NHS was in a flowing state, while the mixture with NHS was completely coagulated. This indicates that NHS facilitates EDC to crosslink gelatin/sodium alginate, and the use of NHS can reduce the amount of EDC used in the synthesis of the gel material.

### Example 10

This example explored the effect of the linking macromolecule content in the hydrogel on the water solubility and enzymatic stability of the gel material.

Referring to Example 1 for the preparation of the material of the present invention, in the experiment, the solution of gelatin 20% (w/w) and the solutions of sodium alginate with different concentrations were prepared using 50 mM MES (pH 6.0). Then, sodium alginate in the solutions was activated with EDC for half an hour. After that, the gelatin solution and the activated sodium alginate solution were mixed in equal volumes. In the final mixed solutions, the concentrations of gelatin and EDC were 10% and 1%, respectively, and the concentration of sodium alginate was 0, 0.5%, 1%, or 1.5%.

500 μL of a freshly prepared gel mixed solution was placed onto a polytetrafluoroethylene plate, which was kept in a humidity chamber at room temperature overnight and weighed. Then, the gel samples with every sodium alginate content were randomly divided into three groups:
the gel samples with sodium alginate concentration of 0 were divided into three groups,
the gel samples with sodium alginate concentration of 0.5% were divided into three groups,
the gel samples with sodium alginate concentration of 1% were divided into three groups, and
the gel samples with sodium alginate concentration of 1.5% were divided into three groups.

In the first group, Control group, the samples were air-dried at room temperature and weighed.

In the second group, Warm water group, the samples were placed in deionized water at 37° C. for 3.5 hours, taken out, air-dried at room temperature, and weighed.

In the third group, Enzymolysis group, the samples were placed in a PBS buffer (pH 7.4) containing 0.1% trypsin, placed at room temperature for 40 minutes, taken out, air-dried at room temperature, and weighed.

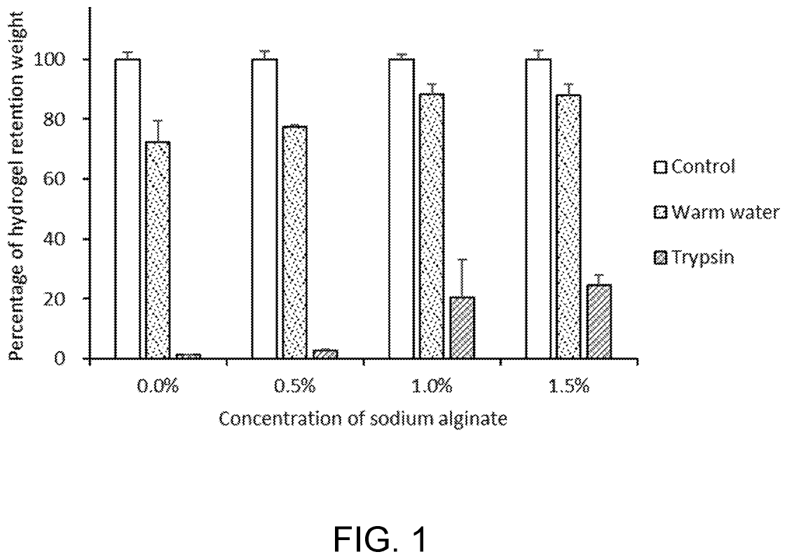

The retention weight percentage of the gel in each group was calculated (Wₜ/W₀×100%); Wₜ is the dry weight of the sample after being placed in deionized water at 37° C. for 3.5 hours or after enzymolysis for 40 minutes, and W₀ is the weight of the gel sample corresponding to the sample with the same sodium alginate content hydrogel, the same initial weight, and after direct drying. The retention weight percentage of the samples in the control group with different sodium alginate contents was taken as 100%. The study found that with increase of sodium alginate content, the retention weight percentage of the gel materials in warm water and trypsin solution increased (FIG. 1), indicating that sodium alginate, as a linking molecule, can enhance the stability of the gel materials.

### Example 11

Using sodium acetate as an example, the experiment explored the effect of adding neutralizing molecules to the reaction system on gelatin crosslinking, and also investigated the effects of one-step synthesis procedure and two-step synthesis procedure on EDC to crosslink gelatin.

10% (w/w) gelatin solution, 5% (w/v) EDC solution, and 100 mM sodium acetate solution (pH 6.0) were prepared using 50 mM MES solution (pH 6.0).

In the one-step synthesis procedure, 50 mM MES solution (pH 6.0), 10% (w/w) gelatin solution, and 100 mM sodium acetate solution (pH 6.0) were first mixed in a certain volume ratio, and then 5% EDC solution was added immediately. In the mixed solutions, the final concentrations of gelatin and EDC were 5% and 0.2%, respectively, and the final concentration of sodium acetate was 0, 5, 10, 20, 30, 40, or 48 mM. After the mixed solution was placed at 37° C. for 2 hours, the flow state of the mixed solution was observed;

In the two-step synthesis procedure, 50 mM MES solution (pH 6.0), a 100 mM sodium acetate solution (pH 6.0), and a 5% EDC solution were first mixed in a certain volume ratio, and the mixture was allowed to stand at room temperature for 1 hour, and then mixed with the same volume of 10% gelatin solution. In the mixed solutions, the final concentrations of gelatin and EDC were 5% and 0.2%, respectively, and the final concentration of sodium acetate was 0, 5, 10, 20, 30, 40, or 48 mM. After the mixed solution was placed at 37° C. for 2 hours, the flow state of the mixed solution was observed.

After the mixed solution was allowed to stand at 37° C. for 2 hours, regardless of whether it was a one-step or two-step synthesis procedure, it was found that with the increase of sodium acetate concentration, the reaction products gradually changed from a coagulated state to a flowing state (Table 1), indicating that sodium acetate can consume EDC; the higher the concentration of sodium acetate, the more EDC is consumed and the less EDC reacts with gelatin, thereby reducing the degree of gelatin crosslinking.

Comparing the one-step synthesis procedure with the two-step synthesis procedure, for the concentration of sodium acetate within the range of 5-40 mM, corresponding to the same concentration of sodium acetate, the product from the two-step synthesis procedure has stronger flowability than the product from the one-step crosslinking procedure. It indicates that in the two-step synthesis procedure, the carboxyl groups of the same concentration can more effectively consume EDC, thereby reducing the concentration of EDC to react with gelatin, and thus reducing the degree of gelatin crosslinking.

This comparative example reveals that the neutralizing agent containing carboxyl groups and the two-step synthesis procedure, in which the crosslinking activator first reacts with the linker to activate the linker into component A, and then mixes it with component B collagen/gelatin, help reduce the amount of EDC in the final reaction system.

TABLE 1
Effects of sodium acetate and the synthesis process on gelatin crosslinking

| | Concentration of sodium acetate (mM) | | | | | | |
|---|---|---|---|---|---|---|---|
| | 0 | 5 | 10 | 20 | 30 | 40 | 48 |
| One-step | Coagulated | Coagulated | Coagulated | Slightly | Slightly | Semi- | Flowing |
| crosslinking | | | | flowing | flowing | flowing | |
| procedure | | | | | | | |
| Two-step | Coagulated | Semi- | Flowing | Flowing | Flowing | Flowing | Flowing |
| crosslinking | | flowing | | | | | |
| procedure | | | | | | | |

### Example 12

### Testing of Tissue Adhesiveness and Tissue Leakage Prevention of the Gel Material in the Present Invention

Excellent tissue healing-promoting materials need to have the ability to adhere closely to the surface of various target tissues. This experiment tested the adhesiveness and tissue leakage prevention function of gel materials.

In the experiment, a hole with 2 mm diameter was first created on the wall of the pig small intestine. Then, the gel material containing 10% gelatin/1% carboxymethyl cellulose prepared in Example 2 was applied to the hole. When the gel coagulated completely, one end of the small intestine was clamped with a hemostatic forceps, and the other end was connected with an inflatable balloon with a pressure gauge. Air was inflated into the small intestine. When the hydrogel blocking the hole in the intestinal wall broke, the highest pressure in the small intestine was recorded, and this pressure was defined as the bursting pressure of the gel.

The reference material is a gel material without carboxymethyl cellulose prepared according to Example 2. The bursting pressure of the reference gel material was 8.1±0.5 kPa, and the bursting pressure of the gel material in Example 2 was 11.8±1.2 kPa. This reveals that the gel material, especially the gel material containing the linker molecule carboxymethyl cellulose, can adhere closely to the tissue surface and has considerable mechanical strength, thus having the function of preventing tissue leakage.

The other examples 1-8 all had mechanical strength comparable to that of Example 2.

### Example 13

### Hemostatic Effect of the Gel Material in the Present Invention

The hemostatic performance of the hydrogel was tested using a rat liver hemorrhage model. Thirty SD rats (male, 170-190 g) without specific pathogen were used. After anesthetized, the rats were fixed on a foam board, and the hair on their abdomen was shaved. Then, a 4 cm incision along the midline of the abdomen was created. The liver was pulled out of the abdominal cavity. A pre-weighed filter paper was placed under the liver, and the left lateral lobe of rat liver was punctured at 30° with an 18G needle to a depth of 10 mm. Immediately afterwards, 0.2 mL of the gel material containing 10% gelatin/1% carboxymethyl cellulose prepared in Example 2, or the reference gel material without carboxymethyl cellulose was applied to the wound. After 3 minutes, the filter paper absorbing blood was weighed.

In the control group, the amount of bleeding was measured 3 minutes after liver injury without using any materials.

In the control group without using any materials for sealing, the liver blood loss was 536±172 mg. Using gel material containing 10% gelatin without carboxymethyl cellulose or with 1% carboxymethyl cellulose, the amount of liver blood loss in rats was significantly reduced to 396±154 mg and 262±115 mg, respectively, revealing that the gel material has a hemostatic function; in particular, the 10% gelatin and 1% carboxymethyl cellulose gel material prepared in Example 2 has better hemostatic performance.

### Example 14

### Cell Compatibility of the Gel Material in the Present Invention

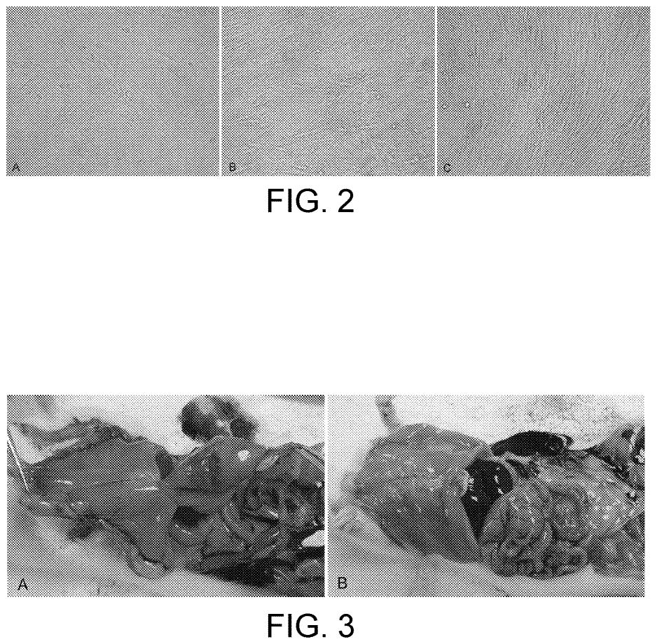

Using the method in Example 3, component A containing 2% sodium hyaluronate, 2% EDC, and 2% NHS, and component B containing 20% gelatin were first prepared. The component A and the component B were filtered through the membrane with the pore diameter of 0.6 μm to remove bacteria that might be present. Then, they were mixed according to the method in Example 3 and injected into the wells of a 24-well cell culture plate, covering the entire bottom of the wells. The plate was allowed to stand at room temperature until the material coagulated. 1×10⁴ mouse fibroblast cells were added in each well. After culturing for 1 day, many cells adhered to the surface of the gel material. As the culture time increased, the cell density gradually increased until the surface of the gel material was completely covered (FIG. 2).

It was demonstrated that the gel material in the present invention has good biocompatibility, and other embodiments also exhibit good biocompatibility.

### Example 15

Prevention of Tissue Adhesion with the Gel Material in the Present Invention

This implementation example is based on a rat model. 20 rats with the same age and in good health condition were taken and divided into two groups, with 10 rats in each group. The following surgical procedures were performed for each rat: the rat was anesthetized, the abdominal skin was cut along the midline of the abdomen, the abdomen was opened along the Linea alba muscle. The cecum was taken outs and brushed with a toothbrush 100 times to cause a punctate hemorrhage injury with an area of about 1 cm×2 cm. A piece of superficial muscle with an area of 1 cm×2 cm was dissected from the inner surface of the abdominal wall, 1 cm away from the midline of the abdomen on the side where the cecum was located.

In the control group, the injured cecum was directly returned to the abdominal cavity with its injured surface close to the injured muscle surface. Then the abdomen was closed, and the muscles and skin were sutured in sequence.

In the gel group, 0.5 ml mixture of component A and component B prepared in Example 5 was uniformly applied to the injured sites of abdominal wall and cecum. After the material coagulated, the injured surfaces of the abdominal wall and cecum were brought close to each other. The abdomen was closed, and then the muscles and skin were sutured in sequence. After 2 weeks of feeding, the rats were euthanized, and the abdominal cavity was opened with a U-shaped incision to observe the adhesion between the abdominal wall and the cecum of the rats.

The study found that adhesions occurred between the abdominal wall and the cecum of all rats in the control group (A of FIG. 3), while no adhesions occurred between the abdominal wall and the cecum of rats in the gel group (B of FIG. 3).

### Example 16

Promotion of Tissue Healing with the Gel Material in the Present Invention

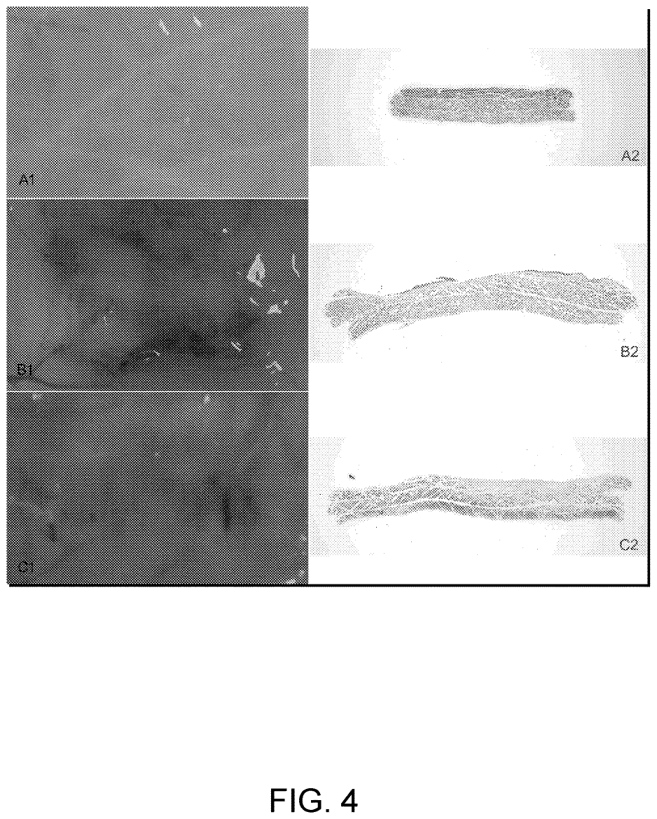

The rats for the adhesion comparison in Example 15 above were euthanized. The abdominal cavity was opened, and the adhered abdominal wall and cecum of the rats were carefully separated. The color and morphology of the injured abdominal muscles and the uninjured muscles on the opposite side of the midline of the abdomen were observed. It was found that the surface of the uninjured abdominal wall muscles of the rats was smooth and ruddy (A1 of FIG. 4); in the control group, the superficial muscle layer of the rat injured abdominal wall showed a significant loss, and there was only a small amount of new tissue in the defect area (B1 of FIG. 4); With the treatment of the gel material prepared in Example 5 in the present invention, the muscle defect surface was covered with a thick layer of new-generated tissue, which was reddish and close to the condition of the normal muscle layer (C1 of FIG. 4).

Uninjured and injured abdominal wall muscles of rats were taken, fixed in formalin, embedded in paraffin, and sectioned into 5 μm thick sections using a histotome. The sections were stained with hematoxylin-eosin and observed under a microscope. It was found that the uninjured abdominal wall muscles of rats consisted of three layers from the outside to the inside (A2 of FIG. 4); after the inner layer of the rat abdomen muscle was dissected and the rats were fed for 2 weeks, the inner muscle layer at the defect was still missing with only a small amount of new matrix in the control group (B2 of FIG. 4); after the inner layer of rat abdominal muscle was dissected, the wound was covered with the gel material, and the defect of inner muscle layer was almost filled after 2 weeks of feeding (C2 of FIG. 4).

The above comparison demonstrates that the gel materials prepared in Examples 1-8 in the present invention have good biocompatibility and can improve the healing of the injured muscle layers.

Obviously, the above examples are only instances for clearly illustrating the present invention, rather than limiting the embodiments of the present invention. For those of ordinary skill in the art, other variations and modifications in different forms can be made based on the above description. It is neither necessary nor feasible to exhaustively list all embodiments here. Any modifications, equivalent substitutions, and improvements made within the spirit and principles of the present invention shall fall within the scope of protection of the claims of the present invention.

What is claimed is:

1. An injectable hydrogel material formed in-situ, comprising two fluid components: component A and component B, wherein the component A is an activated linker solution with a pH value of 4-8, and the component B is a collagen and/or gelatin fluid with a pH value of 4-9;
the component A and the component B exist independently before use, and during use, the component A and the component B are mixed and chemically crosslinked to form the hydrogel material.

2. The injectable hydrogel material formed in-situ according to claim 1, wherein a linker in the component A comprises any one or a mixture of hyaluronic acid, alginate, sodium carboxymethyl cellulose, sodium carboxymethyl starch, and derivatives thereof, and the linker has a mass content of 0.01-5% in the injectable hydrogel material.

3. The injectable hydrogel material formed in-situ according to claim 1, wherein an activator in the component A comprises 1-ethyl-3-(3-dimethylaminopropyl) carbodiimide or a hydrochloride thereof, and the activator has a mass content of 0.1-3% in the injectable hydrogel material.

4. The injectable hydrogel material formed in-situ according to claim 1, wherein a preparation method for the component A is as follows:
dissolving a linker and an activator together in a carboxyl-free solvent, controlling the pH value of the solution to 4-8, and activating for the reaction time ≥5 min, to obtain the component A.

5. The injectable hydrogel material formed in-situ according to claim 1, wherein the component B is the collagen and/or the gelatin dissolved or suspended in an aqueous solution to form an injectable fluid, the collagen has a mass content of 0.1-15% in the injectable hydrogel material, and the gelatin has a mass content of 1-30% in the injectable hydrogel material.

6. The injectable hydrogel material formed in-situ according to claim 1, wherein the component B further comprises a neutralizing agent, and the neutralizing agent is an organic molecule containing a carboxyl group or an organic molecule capable of generating a carboxyl group.

7. The injectable hydrogel material formed in-situ according to claim 1, wherein a molar amount of carboxyl groups or carboxyl groups generated from the neutralizing agent in the component B is 0.1-10 times that of the crosslinking activator.

8. The injectable hydrogel material formed in-situ according to claim 1, wherein the component B further contains the chromogenic agent.

9. A tissue repair formulation, wherein it is prepared from the in-situ formed injectable hydrogel material according to claim 1.

10. A tissue repair formulation prepared from the in-situ formed injectable hydrogel material according to claim 1, prepared by the following method:
loading the component A into a syringe 1, loading the component B into a syringe 2, mounting the syringe 1 and the syringe 2 at two inlets of a three-way connector, extruding two fluids simultaneously, and uniformly mixing the two fluids through a mixing tube mounted at an outlet of the three-way connector, or stirring an extruded solution with the outlet of the three-way connector, to make solutions uniformly mixed and solidified to form the tissue repair formulation.
